(19) United States
(12) Patent Application Publication
(10) Pub. No.: US 2006/0204481 A1
(43) Pub. Date: Sep. 14, 2006

(54) Methods of promoting cell viability

(76) Inventor: Clifford J Steer, OSAKA (JP)

Correspondence Address: RATNERPRESTIA, P O BOX 980, VALLEY FORGE, PA, 19482-0980, US

(21) Appl. No.: 10/549,867

(22) PCT Filed: Apr. 2, 2003
(86) PCT No.: PCT/US03/09819

§ 371 Date: Sep. 22, 2005

(30) Foreign Application Priority Data
Oct. 3, 2003 (JP) 2003-345398

(51) Int. Cl. A61K 35/12; A01N 1/02; A61K 31/56

(52) U.S. Cl. 424/93.7; 435/1.1; 514/169

(57) ABSTRACT

Methods for promoting viability of a transplant cell population by administering a hydrophilic bile acid, such as ursodeoxycholic acid (UDCA), salts thereof, and analogs thereof (e.g., glycoursodeoxycholic acid and tauroursodeoxycholic acid).

### BACKGROUND

The transplantation of cells (e.g., organs) is currently in wide use for the treatment of a variety of human diseases. Heart transplantation, for example, is routinely performed for patients with failing hearts. A major obstacle in transplantation, however, is the lack of quality tissue for implantation. Many cells (e.g., organs) are ultimately not implanted because of poor survival after harvesting. Methods to promote cell viability for transplantation are therefore desperately needed.

An example of a disease in which transplantation can be used is Parkinson's disease. Parkinson's disease (PD) is a disorder of the central nervous system that affects between one and one-and-a-half million Americans. PD may appear at any age, but the risk for developing PD increases with age. In addition, PD occurs in all parts of the world, and affects men slightly more often than women. The symptoms of PD may include rigidity, tremors, bradykinesia, difficulties in walking, and/or difficulties with balance. People with PD usually do not experience all of these symptoms, but rather a subset of these symptoms.

The actual cause of PD is not known. It is believed that PD results from a combination of genetic predisposition and an as yet unidentified environmental trigger. When PD occurs, degenerative changes are found in the area of the brain known as the substantia nigra, which produces dopamine. Dopamine is a chemical substance that enables people to move normally and smoothly. PD is characterized by a severe shortage of dopamine. It is believed that the deficiency of dopamine causes the symptoms of PD.

Transplantation of human embryonic dopamine neurons in subjects with PD is currently being evaluated in clinical trials (A. Björklund et al., Nat. Neurosci. 3:537-544, 2000.). Although it has been reported that a small number of subjects with PD exhibit a normalization of dopamine content in the brain and return to a normal life after neural transplantation (J. H. Kordower et al., J. Comp. Neurol. 370:203-230, 1996; P. Piccini et al., Nat. Neurosci. 2:1137-1140, 1999), the majority of transplanted subjects show incomplete symptomatic recovery (C. R. Freed et al., N. Engl. J. Med. 344:710-719, 2001; Lindvall, O., Cell Transpl. 4:393-400, 1995). Several factors may contribute to this limitation. Among them, the loss of transplanted dopamine neurons remains one of the main obstacles to complete clinical improvement. Numerous animal experimental and clinical studies have shown that the survival rate of grafted dopamine neurons is only about 10% when dopamine neurons are transplanted into the brain using a cell suspension technique (P. Brundin et al., Prog. Brain Res. 71:293-308, 1987; N. Nakao et al., Proc. Natl. Acad. Sci. USA 91:12408-12412, 1994; H. Sauer et al., Restor. Neurol. Neurosci. 2:123-135, 1991; H. Sauer et al., Exp. Brain Res. 90:54-62, 1992; G. S. Schierle et al., Nat. Med. 5:97-100, 1999; A. Zuddas et al., Eur. J. Neurosci. 3:72-85, 1991).

In a recent review, it has been estimated that a minimum of 80,000 dopamine neurons is required for manifested clinical benefits (A. Björklund et al., Nat. Neurosci. 3:537-544, 2000). However, several clinical studies have demonstrated that the number of surviving dopamine neurons in the brain is far smaller than this number in subject specimens (C. R. Freed et al., N. Engl. J. Med. 344:710-719, 2001). The enhancement of dopamine neuron survival therefore becomes a pertinent issue (P. Brundin et al., Cell Transplant. 9:179-195, 2000). In addition, the improvement of dopamine neuron survival can circumvent the limited availability of human embryonic dopamine tissue that is an obstacle in the clinical application of neural transplantation.

There is substantial evidence that apoptosis plays a role in the loss of transplanted dopamine neurons (M. Emgard et al., Exp. Neurol. 160, 279-288, 1999; T. J. Mahalik et al., Exp. Neurol. 129:27-36, 1994; G. S. Schierle et al., Nat. Med. 5:97-100, 1999; W. M. Zawada et al., Brain Res. 786, 96-103, 1998). In addition, apoptosis normally takes place within the first several days after transplantation (M. Emgard et al., Exp. Neurol. 160, 279-288, 1999; G. S. Schierle et al., Nat. Med. 5:97-100, 1999; W. M. Zawada et al., Brain Res. 786, 96-103, 1998). In an in vitro study, apoptosis associated with the loss of dopamine neurons was found to occur predominantly during the first 24 hours, and about 50% of the dopamine neurons were already lost in the first 8 hours (R. L. Branton et al., Exp. Neurol. 160:88-98, 1999). These studies provide possible explanations to the previous observations that the majority of cell death in nigral grafts occurs immediately after transplantation (R. A. Barker et al., Exp. Neurol. 141:79-93, 1996; W.-M. Duan et al., Exp. Brain Res. 104:227-242, 1995).

As neural transplantation of human embryonic dopamine neurons in subjects with PD have shown incomplete symptomatic recovery, there continues to be a need for reducing the loss of transplanted cells in subjects. This need applies to not only neurons for PD, but to other transplant applications.

### SUMMARY

The present invention provides a method for promoting the viability of a transplant cell population to be transplanted into a subject. Preferably the subject is a human subject, and promoting viability of a transplant cell population includes contacting the transplant cell population with an effective amount of a compound selected from the group consisting of a hydrophilic bile acid, a salt thereof, an analog thereof, and a combination thereof.

The cells of the transplant cell population can include, for example, differentiated cells and precursor cells. In addition, the cells of the transplant cell population can include autologous cells, heterologous cells, or xenologous cells. Alternatively, the cells of the transplant cell population can include at least a portion of autologous tissue, heterologous tissue, or xenologous tissue. For certain embodiments the cell population is in the form of an organ, or a part thereof, such as a liver, heart, kidney, lung, and pancreas, for example. Preferably, the cell population is a human cell population.

The contacting can occur in vitro, in vivo, and a combination thereof. As used herein, in vitro is to be distinguished from in vivo. In vitro refers to an artificial environment location of the transplant cell population to be treated, such as a cell culture in a tissue culture dish. In vivo refers to a natural environment location of the cell population to be treated, such as in a mammalian body.

One aspect of the present invention provides a method that includes contacting the transplant cell population with the compound prior to transplanting the transplant cell population in a subject. This can be done prior to (in vivo) or after (in vitro) removal of the transplant cell population from the donor. Alternatively, this can be done after the transplant cell population has been transplanted in the subject.

In addition or alternatively, the present invention provides a method that includes treating the subject to receive the transplant cell population with the compound. For example, treating the subject with the compound can include administering the compound to the subject prior to transplanting the transplant cell population in the subject. In addition, as mentioned above treating the subject with the compound can include administering the compound to the subject after transplanting the transplant cell population in the subject. In this latter example, the subject may have already been treated with the compound prior to transplanting the transplant cell population in the subject. Preferably, treating the subject includes treating the subject parenterally or orally with the compound.

The present invention also provides for treating a donor, and/or a subject, of a transplant cell population with a compound selected from the group consisting of a hydrophilic bile acid, a salt thereof, an analog thereof, and a combination thereof. As discussed herein, a hydrophilic bile acid useful for the present invention can include, but is not limited to, ursodeoxycholic acid and/or tauroursodeoxycholic acid.

One aspect of the present invention provides a method that includes treating the donor of the transplant cell population with the compound. This can be done prior to (in vivo), during (in vivo) or after (in vitro) removal of the transplant cell population from the donor. Alternatively or in addition to, the present invention provides a method that includes treating the subject of the transplant cell population with the compound. This can be done prior to (in vivo/in vitro), during (in vivo) or after (in vivo) transplanting the transplant cell population into the subject.

Another aspect of the present invention is a method for treating a subject, preferably a human, having a disease that requires cell replacement. For example, the present invention can provide a method for treating a subject, preferably a human, having Parkinson's disease. The method can include contacting a transplant cell population with an effective amount of a compound selected from the group consisting of ursodeoxycholic acid, a salt thereof, an analog thereof, and a combination thereof to promote viability of the transplant cell population. The method then further includes transplanting the transplant cell population into the subject.

An example of an analog of ursodeoxycholic acid includes a conjugated derivative, where the conjugated derivative can be tauroursodeoxycholic acid. In a specific example, the method of the present invention can include treating a human having Parkinson's disease, where the method includes contacting a transplant cell population in vitro with an effective amount of the tauroursodeoxycholic acid in combination with a pharmaceutically acceptable carrier, where the transplant cell population are differentiated cells. The method further includes transplanting the transplant cell population into the human.

As mentioned above, the transplant cell population for the human having Parkinson's disease can include differentiated cells and precursor cells. In addition, the cells of the transplant cell population can include autologous cells, heterologous cells, or xenologous cells. Alternatively, the cells of the transplant cell population can include at least a portion of autologous tissue, heterologous tissue, or xenologous tissue. The contacting step can occur in vitro, in vivo, and a combination thereof. In one embodiment, the cell population is a human cell population.

### BRIEF DESCRIPTION OF THE DRAWINGS

FIG. 1 is a bar diagram summarizing the effects of TUDCA (50 μM/ml) on the survival of TH-positive neurons (shadow bars) and total cells (open bars) in ventral mesencephalic (VM) tissue cultures. Bars represent the mean±S.E.M. of three independent experiments with quadruplicate wells for each culturing condition. †*p<0.01, significant difference from the 7 DIV+TUDCA cultures (one-factor ANOVA with post-hoc Scheffé's F-test).

FIG. 2 is a bar diagram illustrating the effects of TUDCA (50 μM/ml) on apoptosis in VM tissue cultures. Bars represent the mean±SEM of three independent experiments with quadruplicate wells for each culturing condition. *p<0.01, significant difference from the 2 DIV and 7 DIV+TUDCA cultures (one-factor ANOVA with post-hoc Scheffé's F-test).

FIG. 3 is a bar diagram illustrating net ipsilateral amphetamine-induced rotation asymmetry (full turns per minute (min) contralateral to the lesion subtracted from turns ipsilateral to the lesion) over the 90-min test session for the control and the TUDCA-treated groups. The rats were tested before grafting and 2 and 6 weeks after grafting. Each bar represents the group mean and the error bars denote S.E.M.*p<0.01 (paired Student t-test) when compared to pregrafting value and p<0.05 (one-factor ANOVA with post-hoc Scheffé's F-test) when compared to the control group. The symbols †‡ p<0.01 (paired Student t-test) when compared to pregrafting values.

FIGS. 4A-4E are photomicrographs of coronal sections through the grafted striatum processed for TH-immunocytochemistry. The photographs illustrate typical grafts from a representative rat in the TUDCA-treated (A, C, and E) and control (B and D) groups six weeks after transplantation. (A) and (B) demonstrate a overview of grafted brain at a low magnification. The host striatal areas adjacent to the grafts are reinnervated by TH-immunopositive fibres from transplanted neurons. Scale bars in B=1 mm, in D=100 μm and in E=25 μm.

FIG. 5 is a bar diagram of the mean number of TH-immunopositive cells and the graft volume in the control and TUDCA-treated groups six weeks after transplantation. The bars represent the group mean value+S.E.M.*p<0.01 (one-factor ANOVA with post-hoc Scheffé's F-test) when compared to the TUDCA-treated group.

FIG. 6 is a bar diagram illustrating the mean number of apoptotic cells in the graft areas for the control and TUDCA-treated groups 4 days post-transplantation. The number of apoptotic cells in the graft areas was significantly smaller in the TUDCA-treated group than in the control group. An asterisk indicates p<0.01 (one-factor ANOVA with post-hoc Scheffé's F-test) compared to the control group. Error bars represent S.E.M.

### DETAILED DESCRIPTION OF ILLUSTRATIVE EMBODIMENTS

At present, there is a need for an effective treatment to promote the viability of a transplant cell population for treating various medical conditions. The present invention provides such a method for promoting the viability of a transplant cell population. The method can include contacting the transplant cell population with an effective amount of a compound selected from the group consisting of a hydrophilic bile acid, a salt thereof, an analog thereof, and/or a combination thereof prior to, during, or after transplanting the transplant cell population into a subject.

As used herein “promoting” transplant cell population viability includes maintaining, prolonging and/or improving the survival and/or proliferation of the transplant cell population to be transplanted, or those that have been transplanted, into the subject. Further, the term “viability” refers to maintaining the normal function of cells in a transplant cell population, typically in vivo, however the term is meant to include in vitro as well. The term “prolonging” means that transplant cell population for transplantation are preserved by treatment using the method of the invention as compared to a similar transplant cell population that has not been so treated. While not wanting to be bound by a particular theory, it is believed that contacting the transplant cell population for transplantation with the compound of the present invention inhibits programmed cell death, thereby prolonging the viability of the transplant cell population.

As used herein, “transplant cell population” includes, but is not limited to, a population of individual cells and cells present in tissue and/or an organ that has been or can be transplanted into a subject. As such, the cells of the transplant cell population could include matrix structures (e.g., proteins, polysaccharides, peptides, and other molecules) found in tissues and/or organs. As used herein “a,” “an,” “the,” “at least one,” and “one or more” are used interchangeably.

Subjects having a medical condition that can benefit by improved viability of a transplant cell population includes those with neurodegenerative diseases such as Parkinson's disease and Alzheimer's disease; Huntington's disease; multiple sclerosis; amyotrophic lateral sclerosis; cerebellar ataxia; lysosomal storage disorders; cancer; birth defects; those in need of organ and/or tissue transplants; spinal cord injury; ischemic injury such as stroke, ischemic kidney disease, and heart disease; burns; autoimmune diseases; diabetes; inflammatory diseases such as osteoarthritis and rheumatoid arthritis, to name only a few. This list, however, is not exhaustive and other medical conditions known to benefit from improved viability of the transplant cell population to be transplanted are also considered to be within the scope of the present invention.

With respect to measuring the promotion of viability of a transplant cell population, as defined herein, assessments can be made by measurements of ATP levels and capacity for protein synthesis for the cells in the transplant cell population. ATP levels impact energy production during and following transplantation. Capacity for protein synthesis is a general indicator of cellular viability because it requires the integration of several complex biochemical pathways.

Preferably, the transplant cell population can include a transplant cell population obtained from a donor for transplantation into a subject. The transplant cell population may be derived from children, adults, or fetal tissue. Preferably, the transplant cell population can include, but is not limited to, a population of cells derived from blood and all of its components, including erythrocytes, leukocytes, platelets, serum, hematopoietic stem cells; central nervous tissue, including brain and spinal cord tissue, neurons, glia, and neural stem cells; peripheral nervous tissue, including ganglia, posterior pituitary gland, adrenal medulla, and pineal; connective tissue, including skin, skin stem cells, ligaments, tendons, and fibroblasts; muscle tissue, including skeletal, skeletal muscle stem cells, smooth, and cardiac tissues or the cells therefrom; endocrine tissue, including anterior pituitary gland, thyroid gland, parathyroid gland, adrenal cortex, pancreas and its subparts (e.g., islet cells), testes, ovaries, placenta, and the endocrine cells that are a part of each of these tissues; blood vessels, including arteries, veins, capillaries, and the cells from these vessels; lung tissue; heart tissue; brain tissue; heart valves; liver; kidney; intestines; bone; immune tissue, including blood cells, bone marrow, and spleen; fat tissue, including adult progenitor cells (e.g., adult stem cells) derived from fat tissue; eyes and their parts; reproductive tract tissues; and urinary tract tissue. Whole organs are the typical form of the transplant cell population.

As used herein the term “organ” is meant to include all or part of intact multi-cellular organs such as kidney, liver, brain, or heart; cell suspensions derived from multi-cellular organs; as well as suspensions of blood cells or hematopoietic precursor cells. As used herein “tissue” is meant to include aggregations of a transplant cell population with their associated intracellular matrix (e.g., collagen, proteins, polysaccharides, etc.). The transplant cell population can also include individual cells that have been separated and/or isolated from the tissue and/or organ from which they were derived.

With respect to a transplant cell population that includes separated and/or isolated cells, the transplant cell population for transplantation into a subject can include differentiated cells and/or precursor cells (e.g., undifferentiated cells). By way of example, differentiated cells can include, but are not limited to, the cells discussed herein, including but not limited to, myocardial cells, muscle cells, smooth muscle cells, epidermal cells, neurons including dopamine neurons, pancreatic cells, bone marrow cells, hepatic and nonhepatic cells. By way of example, precursor cells can include, but are not limited to, stem cells, pluripotent stem cells, embryonic stem cells, and adult stem cells.

Preferably, cells of the transplant cell population useful with the present invention include, but are not limited to, autologous cells, heterologous cells, xenologous cells, or combinations thereof. As discussed above, cells can also include those cells that form a portion of tissues and/or organs. So, for the present invention, the cells can include at least a portion of autologous tissue, heterologous tissue, xenologous tissue, and/or combinations thereof, wherein the tissue can be transplanted into the subject.

As used herein, “subjects” can include mammals. Preferably, a subject as used herein is a human. As used herein “donor” can include mammals used as a source of biological material, such as a part of, or all of, a cell population (including organs) for transplanting into a subject. The donor can include, but is not limited to, a human, a porcine, a non-human primate, a bovine, or a combination thereof. In addition, the donor can be living during the donation of the cell population.

Further, it is understood that a transplant cell population as defined herein for transplantation can be derived from any species. The present invention is useful for preserving a transplant cell population for use in same species transplant in a subject such as human and other human donors (allografts and autologous or heterologous grafts) or to a human subject from another species such as sheep, pig, cow, or non-human primate (xenografts), for example. Such a transplant cell population for transplant includes, but is not limited to, heart, liver, kidney, lung, pancreas, pancreatic islets, brain, cornea, bone, intestine, skin, blood, and cells from such organs and tissues.

Preferably, contacting the transplant cell population with an effective amount of one or more compounds of the present invention can be accomplished in vitro and/or in vivo. For example, the transplant cell population can be contacted with a compound according to the present invention in vivo prior to transplanting the transplant cell population in the subject. One preferred way of accomplishing this includes treating the donor of a transplant cell population with one or more compounds of the present invention.

In one aspect of the present invention, the donor of the transplant cell population can be treated with the compound of the present invention prior to (in vivo), during (in vivo) or after (in vitro) removal of the transplant cell population from the donor. So, the transplant cell population could be contacted with a compound of the present invention while still in the donor (in vivo). Alternatively, the transplant cell population could be contacted with a compound of the present invention during removal, or once the transplant cell population is removed from the donor. For a donor of a partial organ, for example, benefit to the donor may be realized by administering the compounds of the present invention before and/or after removal of a part of the organ. Preferably, the transplant cell population is perfused and/or immersed or otherwise contacted with a compound of the present invention during harvesting, storing, growing and/or transplanting of the transplant cell population.

In addition, a compound of the present invention can be used in vivo to treat the subject receiving the transplant cell population. This can be done prior to (in vivo/in vitro), during (in vivo) or after (in vivo) transplanting the transplant cell population into the subject. So, it is possible that a subject receiving a transplant cell population also receives treatment with a compound of the present invention. For example, the subject could be treated systemically or locally with the compound by administering the compound of the present invention to the subject prior to (i.e., pre-cell population transplant), during (i.e., concurrent with the time of transplanting the transplant cell population), and/or after (i.e., post-cell population transplant) the transplantation of the transplant cell population.

In an additional example, the transplant cell population can be contacted with a compound according to the present invention in vivo after transplanting the transplant cell population in the subject. So, the transplant cell population could be contacted with a compound of the present invention once the transplanted cell population has been implanted into the subject. In addition, it is possible to use a compound of the present invention in different combinations of in vivo/in vitro treatment regimens as discussed herein.

In a specific example, beginning at a predetermined time prior to the transplantation, the subject may be dosed with immunosuppressive pharmaceuticals to enhance graft acceptance. Immediately prior to transplantation, the subject may be dosed with one or more compounds of the present invention. The donor heterologous transplant cell population can then be flushed with a solution containing at least one compound of the present invention, e.g., tauroursodeoxycholic acid (TUDCA). Following transplantation by standard surgical techniques, the subject is typically maintained on routine immunosuppression pharmaceuticals and optionally, a compound of the present invention. Based on clinical signs and symptoms related to immune responsiveness, the immunosuppressants can be reduced in dosage.

The term “effective amount” as used herein includes useful dosage levels of the compound of the present invention that will be effective to promote the viability of a transplant cell population. Although the inventors do not wish to be bound by theory, it is believed that an “effective amount” is one effective to prevent, reduce, inhibit, or suppress apoptosis of cells to be transplanted and/or cells that have been transplanted. Useful dosages of the desired compound described herein can be determined by comparing its in vitro activity and its in vivo activity in animal models. Methods for extrapolation of effective dosages in mice, and other animals, to humans are known in the art.

It will be understood, however, that the specific “effective amount” for any particular subject (or donor) and/or transplant cell population will depend upon a variety of factors including the activity of the specific compound employed; the transplant cell population; the conditions under which the transplant cell population are being harvested, isolated, stored, and/or incubated when the transplant cell population is maintained in vitro; and when used in vivo, the age, body weight, general health, sex, diet, time of administration, route of administration, rate of excretion, drug combination, and the severity of the medical condition for the subject (or donor) being treated.

Preferably, the transplant cell population can be contacted with a compound of the present invention during storage. Storage of a transplant cell population can include conditions under which the transplant cell population is maintained. Alternatively, storage of the transplant cell population can also include conditions under which the transplant cell population is placed in order to encourage the growth and proliferation of the transplant cell population. In either situation, the transplant cell population can be contacted with an effective amount of a compound of the present invention.

For enhancing transplant cell population survival prior to transplantation, a compound of the present invention may be used with cell culture media used in the culture of the cells to be transplanted into the subject. Concentration of a compound will depend on a variety of factors, including solubility and activity.

Preferably, the methods of the present invention involve the use of a hydrophilic bile acid, salts thereof, analogs thereof, or combinations thereof. As used herein, hydrophilic bile acids are those more hydrophilic than deoxycholic acid (DCA). This can be determined by evaluating the partition coefficient between water and octanol, with the more hydrophilic bile acids being more favorable toward water. Alternatively, the more hydrophilic bile acids have earlier retention times on a reverse-phase column using high performance liquid chromatography. A particularly preferred hydrophilic bile acid includes ursodeoxycholic acid. Examples of analogs of hydrophilic bile acids include conjugated derivatives of bile acids. Although all hydrophilic bile acids may not be useful in all methods of the present invention, they can be evaluated readily by testing their ability to inhibit apoptosis in cell cultures using agents known to induce apoptosis. Two particularly preferred conjugated derivatives include glyco- and tauroursodeoxycholic acid.

Ursodeoxycholic acid (UDCA) is an endogenous bile acid that has been in clinical use over the last few decades for the treatment of a variety of liver diseases. Conjugated derivatives of UDCA include ursodeoxycholic acid 3-sulfate, ursodeoxycholic acid 7-sulfate, ursodeoxycholic acid 3,7-disulfate, tauroursodeoxycholic acid (TUDCA), and glycoursodeoxycholic acid.

Typically, for preferred embodiments, the compound described herein can be formulated in pharmaceutical compositions. Then, in accordance with methods of the invention, a transplant cell population can be then contacted with the pharmaceutical composition containing a compound of the present invention. In addition, the pharmaceutical composition containing a compound of the present invention can be administered to a subject, typically a mammal such as a human subject, in a variety of forms adapted to the chosen route of administration. The formulations include those suitable for in vitro cell culture as well as, oral, rectal, vaginal, topical, nasal, ophthalmic, parenteral (including subcutaneous, intramuscular, intraperitoneal, intravenous, intrathecal, intraventricular, direct injection into brain tissue, etc.) administration.

The formulations may be conveniently presented in unit dosage form and may be prepared by any of the methods well known in the art of pharmacy. Typically, such methods include the step of bringing the active compound into association with a carrier, which can include one or more accessory ingredients. In general, the formulations are prepared by uniformly and intimately bringing the active compound into association with a liquid carrier, a finely divided solid carrier, or both, and then, if necessary, shaping the product into a desired formulation.

Formulations of the present invention suitable for oral administration may be presented as discrete units such as tablets, troches, capsules, lozenges, wafers, or cachets, each containing a predetermined amount of the apoptosis limiting compound as a powder, in granular form, incorporated within liposomes, or as a solution or suspension in an aqueous liquid or non-aqueous liquid such as a syrup, an elixir, an emulsion, or a draught.

The tablets, troches, pills, capsules, and the like may also contain one or more of the following: a binder such as gum tragacanth, acacia, corn starch, or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The compound may be incorporated into sustained-release preparations and devices if desired.

A compound suitable for use in the methods of the invention can also be incorporated directly into the food of a subject's diet, as an additive, supplement, or the like. Thus, the invention further provides a food product. Any food can be suitable for this purpose, although processed foods already in use as sources of nutritional supplementation or fortification, such as breads, cereals, milk, and the like, are convenient to use for this purpose.

Formulations suitable for parenteral administration conveniently comprise a sterile aqueous preparation of the desired compound, or dispersions of sterile powders comprising the desired compound, which are preferably isotonic with the blood of the subject. Isotonic agents that can be included in the liquid preparation include sugars, buffers, and salts such as sodium chloride. Solutions of the desired compound can be prepared in water, optionally mixed with a nontoxic surfactant. Dispersions of the desired compound can be prepared in water, ethanol, a polyol (such as glycerol, propylene glycol, liquid polyethylene glycols, and the like), vegetable oils, glycerol esters, and mixtures thereof. The ultimate dosage form is sterile, fluid, and stable under the conditions of manufacture and storage. The necessary fluidity can be achieved, for example, by using liposomes, by employing the appropriate particle size in the case of dispersions, or by using surfactants. Sterilization of a liquid preparation can be achieved by any convenient method that preserves the bioactivity of the desired compound, preferably by filter sterilization. Preferred methods for preparing powders include vacuum drying and freeze drying of the sterile injectible solutions. Subsequent microbial contamination can be prevented using various antimicrobial agents, for example, antibacterial, antiviral and antifungal agents including parabens, chlorobutanol, phenol, sorbic acid, thimerosal, and the like. Absorption of the desired compounds over a prolonged period can be achieved by including agents for delaying, for example, aluminum monostearate and gelatin.

Nasal spray formulations can include purified aqueous solutions of the desired compound with preservative agents and isotonic agents. Such formulations are preferably adjusted to a pH and isotonic state compatible with the nasal mucous membranes. Ophthalmic formulations are prepared by a similar method to the nasal spray, except that the pH and isotonic factors are preferably adjusted to match that of the eye. Formulations for rectal or vaginal administration may be presented as a suppository with a suitable carrier such as cocoa butter, or hydrogenated fats or hydrogenated fatty carboxylic acids.

In addition, a compound of the present invention can be modified by appropriate functionalities to enhance selective biological properties. Such modifications are known in the art and include those which increase biological penetration into a given biological system (e.g., blood, lymphatic system, central nervous system, brain), increase oral availability, increase solubility to allow administration by injection, alter metabolism and alter rate of exertion. In addition, a compound may be altered to pro-drug form such that the desired compound is created in the body of the subject as the result of the action of metabolic or other biochemical processes on the pro-drug. Some examples of pro-drug forms include ketal, acetal, oxime, and hydrazone forms of a compound that contains ketone or aldehyde groups.

When a compound of the present invention can be delivered in vivo, the dosage level of the compound can be dependent on the route of delivering the compound. For example, when delivered orally the dosage level of the compound of the present invention is preferably on the order of about 10 milligrams or greater, 15 milligrams or greater, or 50 milligrams or greater per kilogram of body weight per day. Also, in a preferred embodiment the dosage level of compound of the present invention for oral delivery is preferably on the order of about 100 milligrams or less, 50 milligrams or less, or 15 milligrams or less per kilogram of body weight per day. Preferably, the dosage level of compound of the present invention for oral delivery has a range on the order of about 10 milligrams to about 100 milligrams per kilogram of body weight per day. In one example, the effective amount by oral administration is on the order of about 500 milligrams to about 1000 milligrams per subject per day.

In an additional example, when the compounds of the present invention are delivered intravenously, the dosage level of the compound of the present invention is preferably even higher than those delivered orally. For example, the dosage level of the compound of the present invention when delivered intravenously can be on the order of about 10 milligrams or greater, 15 milligrams or greater, 50 milligrams or greater, or 100 milligrams or greater per kilogram of body weight per day. Also, in a preferred embodiment the dosage level of compound of the present invention for intravenous delivery is preferably on the order of about 200 milligrams or less, 100 milligrams or less, 50 milligrams or less, or 15 milligrams or less per kilogram of body weight per day. Preferably, the dosage level of compound of the present invention for intravenous delivery has a range on the order of about 10 milligrams to about 200 milligrams per kilogram of body weight per day.

Dosage levels greater than or less than those recited herein are also possible. When a compound of the present invention is delivered to a subject, the compound can be delivered in one or multiple dosages for injection, infusion, and/or ingestion.

One example of where promoting the viability of a transplant cell population for transplanting into a subject would be beneficial is in the treatment of subjects with Parkinson's disease (PD). In PD, degenerative changes are found in the substantia nigra. The substantia nigra is an area of the brain that produces dopamine, a chemical substance that enables people to move normally and smoothly. PD is characterized by a severe shortage of dopamine. It is believed that the deficiency of dopamine causes the symptoms of PD. Although the compounds described herein are believed to play a role in modulating the apoptotic threshold in both hepatic and nonhepatic cells, it was unexpected that they could be used for the treatment of PD because of the unknown origin of the degenerative changes of the substantia nigra. Similarly, it was unexpected that they could be used in promoting viability of a transplant cell population because of concerns of apoptosis of the transplant cell population during the time necessary for engraphment of the transplanted cell population. In other words, it was surprising that the compounds, and the amount of compounds used, of the present invention were able to inhibit apoptosis of the transplanted cell population long enough for engraphment of the transplant cell population to take place in the subject. In addition, it was unexpected that they could be used in promoting viability of the transplant cell population because of the unknown causes that are involved with the loss of transplanted dopamine neurons.

Substantial evidence indicates that apoptosis plays a role in the loss of dopamine neurons in vitro and in vivo. Thus, one example where the present invention can be useful in maintaining the viability of a cell to be transplanted into a subject would be in administering an effective amount of hydrophilic bile acid, a salt thereof, an analog thereof, or a combination thereof, to block apoptotic pathways in the cultures and in the grafts, leading to the enhancement of dopamine neuron survival and the improvement of nigral graft function. Anti-apoptotic agents can be applied to the preparation of transplant cell population prior to transplantation or during the first few days after transplantation to prevent apoptosis and improve graft survival.

Preferably, ursodeoxycholic acid (UDCA), salts thereof, analogs thereof (e.g., conjugate derivative tauroursodeoxycholic acid (TUDCA)), and combinations thereof are useful in promoting viability of a transplant cell population used in treating subjects with PD. For instance, a suspension of human embryonic dopamine neurons obtained by standard methods in the art can be treated by the method of the invention and can be used for neural transplantation in a subject. As an in vitro treatment for example, human dopamine neurons (autologous reconstitution) or derived from an individual other than the subject (heterologous reconstitution) can be expanded and used in the treatment or prevention of PD. As disclosed herein, TUDCA displays anti-apoptotic properties, where supplementation of TUDCA to cell suspensions prior to transplantation can lead to enhanced survival of nigral grafts. This demonstrates that pre-treatment of the cell suspension with TUDCA can reduce apoptosis and increase the survival of grafted cells, resulting in an improvement of behavioral recovery.

Advantages of the invention are illustrated by the following examples. However, the particular materials and amounts thereof recited in these examples, as well as other conditions and details, are to be interpreted to apply broadly in the art and should not be construed to unduly limit the invention.

### EXAMPLES

In the following examples, the role of hydrophilic bile acid on apoptosis of cells in neural graphs is further characterized and the nature and mechanisms of tauroursodeoxycholic acid (TUDCA)-induced neuroprotection delineated. Specifically, TUDCA treatment led to a marked reduction in dopamine neuronal cell death and degeneration. Further, deoxynucleotidyl transferase (TdT)-mediated 2′-deoxyuridine 5′-triphosphate (dUTP)-biotin nick end labeling (TUNEL) assays showed that the number of apoptotic cells in the TUDCA-treated cultures was dramatically smaller than that in the control cultures.

Additionally, cell suspension with TUDCA exhibited a significant reduction in amphetamine-induced rotation scores when compared to pre-transplantation value. There was a significant increase (approximately 3-fold) in the number of tyrosine hydroxylase (TH)-positive cells in the neural grafts for the TUDCA-treated group when compared to the controls 6 weeks post-grafting. The number of apoptotic cells was much smaller in the graft areas in the TUDCA-treated groups than in the control group 4 days after transplantation. These data demonstrate that pre-treatment of the cell suspension with TUDCA can reduce apoptosis and increase the survival of grafted cells, resulting in an improvement of behavioral recovery.

### Materials and Methods

Experimental Design

For the present invention, cell culture and transplantation experiments were conducted. In both the in vitro and in vivo studies, ventral mesencephalic (VM) tissue was dissected from Sprague-Dawley (SD) (Charles River Labs, Wilmington, Mass.) rat embryos at embryonic day 14 under sterile conditions as described (A. Björklund et al., Acta. Physiol. Scand. Suppl. 522:9-18; 1983; P. Brundin et al., In: Conn, P. M. ed. Methods in Neurosciences, Lesions and transplantation. Academic Press, San Diego, Vol 7, 1991:305-326; E. M. Grasbon-Frodl et al., Brain Res. Bull. 39:341-347; 1996). For each experiment, the harvested tissue pieces were equally divided into two experimental groups: control and TUDCA-treated groups. The study was approved by the Animal Care Committee at the University of Minnesota and conducted under the auspices of Research Animal Resources, a facility approved by the American Association for the Accreditation of Laboratory Animal Care.

Cell Culture Experiments

In order to examine the effects of TUDCA on apoptosis and the survival of dopamine neurons in VM tissue cultures under serum-free conditions and to determine an appropriate dosage of TUDCA, primary neuronal cultures were prepared from the VM tissue of SD rat embryos and incubated with or without the addition of TUDCA (Calbiochem, La Jolla, Calif., free base, dissolved in 0.15M (where “M” is molar concentration) NaHCO₃ buffer). In experiments, the dose-response effects of TUDCA were studied and a concentration of 50 μM (micro-Molar) was found to be appropriate. Therefore, TUDCA was added to a final concentration of 50 μM to the culture medium when the culture medium was switched to serum-free conditions after two days in vitro. Neural survival and apoptosis were determined by counting TH-positive cells and TUNEL-positive cells in the cultures two or seven days in vitro. A total of three separate series of cell culture experiments were performed.

Transplantation Experiment

A total of 24 adult female Sprague-Dawley (SD) rats, weighing about 250 grams (g) at the beginning of the experiments were used as subjects of neural grafts. Among them, 12 rats were subjected to an extensive unilateral 6-hydroxydopamine (6-OHDA) lesion of the mesostriatal dopamine (DA) system and 12 rats were normal.

The 6-OHDA lesioned rats and normal rats were randomly assigned to the TUDCA-treated (n=6 in each group) or the vehicle control (n=6 in each group) group, respectively. The rats were housed 2 per cage under a 12 hour day-night cycle with ad libitum access to food and water. They were maintained and treated in accordance with published National Institutes of Health guidelines.

For the TUDCA-treated rats, a 50 μM concentration of TUDCA was added to the media when nigral tissue was trypsinized and dissociated. Two microliters of cell suspension containing TUDCA were then stereotaxically injected into the striatum of SD rats. For the control animals, the same amount of vehicle solution was added to the media. In the first series of transplantation experiment, normal rats that received neural transplants were sacrificed 4 days post-grafting and brain sections were prepared for the terminal deoxynucleotidyl transferase (TdT)-mediated dUTP-biotin nick end labeling (TUNEL) assay to determine apoptosis in the transplantation areas. In the second series of transplantation experiments, an amphetamine-induced rotation test was used to assess the completeness of the 6-OHDA lesions prior to grafting, and repeated two and six weeks after transplantation to monitor functional effects of the neural grafts. The rats were sacrificed after the last session of the rotational behavioral test and the brain tissue was processed for tyrosine hydroxylase (TH)-immunocytochemistry. Graft survival was assessed by counting the number of TH-positive neurons in the grafts.

Ventral Mesencephalic Tissue Cultures

For each experimental series, pooled VM tissue from 24-32 embryos was incubated in 0.1% trypsin (Sigma, St. Louis, Mo.)/0.05% DNase (Sigma, St. Louis Mo.) at 37 degrees Celsius (° C.) for 20 minutes (min), and mechanically dissociated using a 1 milliliter (ml) Gilson pipette. Following dissociation, the cells were centrifuged at 600 rotation per minute (rpm) for 5 min and the pellet was resuspended in Dulbecco's Modified Eagle's Medium (DMEM) (Gibco, Carlsbad, Calif.). The cell number and viabilities of dissociated cells were assessed with a hemocytometer using trypan blue dye exclusion. The number of 100,000 cells/cm² (178,000 cells per well) were plated on to four-well chamber slides (Nunc, Rochester, N.Y.) precoated with 10 milligram per milliliter (mg/ml) poly-d-lysine (Sigma, St. Louis Mo.). The viabilities of dissociated cells were over 95%. Cell cultures were incubated for 2 days in DMEM supplemented with 10% fetal calf serum at 37° C. in a 95% air/5% CO² humidified atmosphere. After 2 days in vitro, the culture medium was switched to serum-free N2 medium, consisting of DMEM/Ham's F 12(1:1) mixture (Gibco, Carlsbad, Calif.).

At this time point, some cultures were supplemented with TUDCA.

Unilateral 6-Hydroxydopamine Lesion

Two injections of 6-OHDA (hydrochloride salt, Sigma, St. Louis Mo.) were made into the right ascending mesostriatal DA pathway under equithesin anaesthesia (0.3 ml/100 g body wt. i.p.) as described previously (W.-M. Duan et al., Neuroscience 57:261-274; 1993). Briefly, a first injection of 2.5 microliter (μl) of 6-OHDA (3 microgram/microliter (μg/μl), free base, in 0.2 mg/ml ascorbate-saline) was performed at the following coordinates: 4.4 millimeter (mm) caudal to bregma; 1.2 mm lateral to midline; 7.8 mm ventral to the dural surface; with the tooth-bar set at 2.4 mm below the interaural line. A second injection of 2 μl of 6-OHDA was performed at the following coordinates; 4.0 millimeter (mm) caudal to bregma; 0.8 mm lateral to midline; 8.0 mm ventral to the dural surface; with the tooth-bar set at 3.4 mm above the interaural line. The 6-OHDA was infused at a rate of 1 μl/min, and the cannula was left in place for an additional 4 min before it was withdrawn.

Amphetamine-Induced Rotation Tests

Two to three weeks after the lesion surgery, the rats were given 5 milligram per kilogram (mg/kg) of d-amphetamine sulphate (Sigma, St. Louis Mo.) in saline, i.p. and their rotational behavior was monitored in automated rotometer bowls for 90 min (U. Ungerstedt et al., Brain Res. 24: 485-493; 1970). Twelve rats that exhibited a net rotational asymmetry of at least 7 full ipsilateral turns per minute towards the lesioned side were selected and divided into two groups that were balanced according to net rotational asymmetry scores (the number of turns contralateral to the lesioned side subtracted from the number of turns ipsilateral to the lesioned side). The rotational test was then repeated at two different time-points as mentioned in the experimental design. Rats that displayed a reduction in rotational asymmetry to less than 50% of the pre-transplantation value were considered to have functional grafts.

Nigral Tissue Preparation and Transplantation

A cell suspension technique was used to perform the neural transplants as previously described (A. Björklund et al., Acta. Physiol. Scand. Suppl. 522:9-18; 1983; P. Brundin et al., In: P. M. Conn, ed., Methods in Neurosciences, Lesions and Transplantation, Academic Press, San Diego, Vol 7, 1991:305-326). Briefly, VM tissue was obtained from embryos with a crown-to-rump length of 13-14 mm, corresponding to a gestational age of embryonic day 14. Dissection and preparation of the donor tissue were carried out under aseptic conditions in Hank's balanced salt solution (HBSS) (Gibco, Carlsbad, Calif.). Uterine horns were removed by hysterectomy from the animals under deep chloral hydrate anesthesia (250 mg/kg, i.p.) and placed in plastic tubes containing HBSS. The embryos were removed from the uterus and embryonic brains were individually transferred to a petri-dish with a dark background. The VM was dissected out from each brain under a dissection microscope using iridectomy scissors and fine watchmaker's forceps. The dissected pieces were pooled and incubated in 0.1% trypsin (Sigma, St. Louis Mo.)/0.05% DNase (Sigma, St. Louis Mo.)/HBSS at 37 degrees Celsius for 20 min. After rinsing 4-5 times with 0.05% DNase/HBSS, the tissue was gently dissociated to a mixture of single cells and small cellular aggregates using fire-polished Pasteur pipettes with an inner diameter of 0.5-1.0 millimeter. The final solution of the cell suspension was adjusted so that five microliters of HBSS were added for each dissected piece of VM tissue. Before and after transplantation, the viabilities of the cell suspensions were assessed by the trypan blue dye exclusion method. The viabilities of all the cell suspensions in this study were over 95%.

One deposit of two microliters of the cell suspension (containing approximately 100,000 cells, equivalent to one-third of VM) was stereotaxically transplanted into the right striatum of equithesin (3 ml/kg, i.p.) anesthetized subject rats fixed in a Kopf stereotaxic frame. Using a 10 microliter Hamilton microsyringe (Hamilton Co., Reno, Nev.) fitted with a steel cannula (inner diameter=0.25 mm, outer diameter=0.47 mm), injections were made at the following coordinates: 1.0 mm rostral to bregma; 3.0 mm lateral to the midline; 4.5 mm ventral to the dural surface, with the tooth-bar set at zero. The injection was performed over 2 min and the needle was left in place for an additional 2-4 min before retraction.

Immunocytochemistry

The avidin-biotin complex immunoperoxidase technique was used to visualize immunocytochemical staining as described previously (W.-M. Duan et al., Neuroscience 100:521-530; 2000). For the cell culture experiments, cultures were rinsed once with phosphate buffered saline (PBS, 0.2 Molar, pH 7.4), followed by fixation with 4% formaldehyde for 20 min at room temperature. Then cultures were processed for immunocytochemistry. For the transplantation experiments, rats were deeply anesthetized with chloral hydrate at a lethal dose (500 mg/kg, body weight, i.p.) and transcardially perfused with 0.1 Molar PBS followed by cold 4% formaldehyde. The brains were then removed and post-fixed for 4 hours in the same fixative, and placed in 20% sucrose at 4 degree Celsius until they sank. Sections were coronally cut at 30 micrometer thickness on a freezing sliding microtome. Throughout the region of the graft, four adjacent series of sections were collected in four glass vials. The following primary antibodies were used against TH (1:500 Pel-Freez, Rogers AR). Biotinylated goat anti-rabbit (rat-absorbed) immunoglobulins (1:200) (Vector Laboratories, Inc., Burlingame, Calif.) were used as the secondary antibody. Sections were incubated in ABC solution (Vectastain ABC Elite kit, Vector Laboratories Inc.) followed by development with 3,3′-diaminobenzidine solution (Vectastain DAB kit, Vector Laboratories Inc.) to visualize the immunoreactive products. After staining, sections were mounted on superfrost microscope slides (Fisher Scientific, Pittsburgh, Pa.), dehydrated through ascending graded concentrations of alcohol, cleared in xylene, and cover-slipped using DPX mounting medium (Fluka, Switzerland).

Terminal Deoxynucleotidyl Transferase-Mediated dUTP-Biotin Nick End Labeling Assay

TUNEL assay was performed by using an in situ Apoptosis Detection Kit (sold under the trade designation ApopTag) according to the manufacturer's protocol (Intergen, Purchase, N.Y.) as described previously (W.-M. Duan et al., Neuroscience 100:521-530; 2000). Briefly, 3-4 sections containing graft tissue were selected from each grafted animal 4 days post-grafting. They were mounted on superfrost microscope slides (Fisher Scientific, Pittsburgh, Pa.) for the following staining protocol. The sections were first quenched in 3% hydrogen peroxide in PBS to remove endogenous peroxidase, and then subsequently incubated in working strength TdT enzyme and peroxidase-conjugated antibody against digoxigenin solution. 3,3′-diaminobenzidine (Sigma, St. Louis Mo.) was used as a chromogen to visualize the reactive products. After counterstaining with methyl green, the sections were dehydrated in alcohol and cleared in xylene and cover-slipped using Permount mounting medium (Fisher Scientific, Fair Lown, N.J.).

Morphological Assessment

In order to avoid subjective bias in the interpretation of the results, all of the morphological assessments were conducted under a microscope with bright-field illumination on stained sections in a blinded manner (W.-M. Duan et al., Neuroscience 100:521-530; 2000).

Cell Counts

For the cell culture experiments, the number of TH-positive cells and the total cells were assessed at ×20 and ×40 magnification, respectively, with the aid of a 400-μm square, reticule grid as described previously (E. M. Grasbon-Frodl et al., Brain Res. Bull. 39:341-347; 1996). Eight fields were selected in order to sample a representative area of each culture well in a systematic fashion. The sampled areas made up 1-3% of the total area of each well. For the transplantation experiments, TH-positive neurons in the grafts were counted on every fourth section using a 10× objective lens in a Nikon light microscope (Nikon, Japan). Only cells were counted when they exhibit at least one neurite or have a visible nucleus. After measurement of the average diameter of TH-immunoreactive cells with an inserted grid using a 20× objective lens, total numbers of TH-immunoreactive neurons were calculated by multiplying the raw counts with a correction factor (2.8) according to the Abercrombie formula (M. Abercrombie, Anat. Rec. 94:239-247; 1946).

TUNEL-Positive Cell Counts

For the cell culture experiments, the number of TUNEL-positive cells was assessed at ×400 magnification with the aid of a 400-μm square, reticule grid. Six fields were selected from each culture well in a systematic fashion. For the transplantation experiments, TUNEL-positive cells in the graft areas were counted in 3-4 sections per animal. Average number of TUNEL-positive cells was calculated and represented a value for single animals.

Volumetric Analyses

The volume of intrastriatal neural grafts was analyzed using a computer assisted image analysis system as described previously (W.-M. Duan et al., Eur. J. Neurosci. 10:2595-2606; 1998). Briefly, all the TH-immunostained sections with grafts were digitized using a 1× objective lens under a Nikon light microscope (Nikon, Japan) that is connected with a high resolution digital camera (COOLPIX 950, Nikon, Japan). The images were first collected and stored in a CompactFlash card. Then the CompactFlash card was read by using a CompactFlash card reader connected with a Pentium III PC (Dell, Dimension XPS T700r, USA) and the images were analyzed using a software package (Scion Image, Version Beta 4.0.2, Scion Corporation, Frederick, Md.). In each section, the graft was manually outlined on the screen and the surface area measured. The number of pixels was subsequently converted into square millimeters. The graft volume was calculated based on graft area, section thickness and frequency.

Statistical Analysis

Data in the text and figures are expressed as means±SEM. Statistical comparisons were performed parametrically using two-factor analysis of variance (ANOVA); one-factor ANOVA followed a post-hoc Scheffé's F-test or a Student's t-test for a group-wise comparison. Statistical significance level was set at p<0.05.

### Results

Cell Culture Experiments

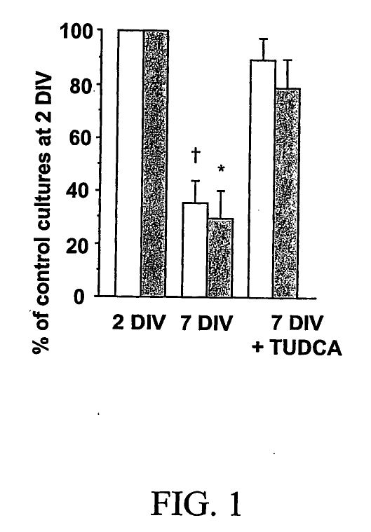

Dopamine neuron survival. At 2 days in vitro, TH-positive cells possessed short processes, indicating that they were in an early developmental stage. Some processes contacted processes from other cells in their vicinity (data not shown). At 7 days in vitro, dopamine neurons exhibited more mature morphology. They sent out several long processes with clear varicosities (data not shown). At this time-point, some of the cell bodies and processes appeared granular under phase-contrast, possibly indicating that they were undergoing degeneration. There was a significant cell loss for TH-positive neurons and total cells in 7 DIV control cultures (p<0.01, one-factor ANOVA with post-hoc Scheffé's F-test). Indeed, the number of TH-immunopositive neurons was only 30% of the value in 2 DIV cultures. This dramatic cell loss was not observed in 7DIV TUDCA-treated cultures (data not shown). No difference was found in the number of TH-positive cells between 2DIV cultures and TUDCA-treated cultures (p>0.05) (FIG. 1). TUDCA exerted this neural protective effect in a dose-dependent manner.

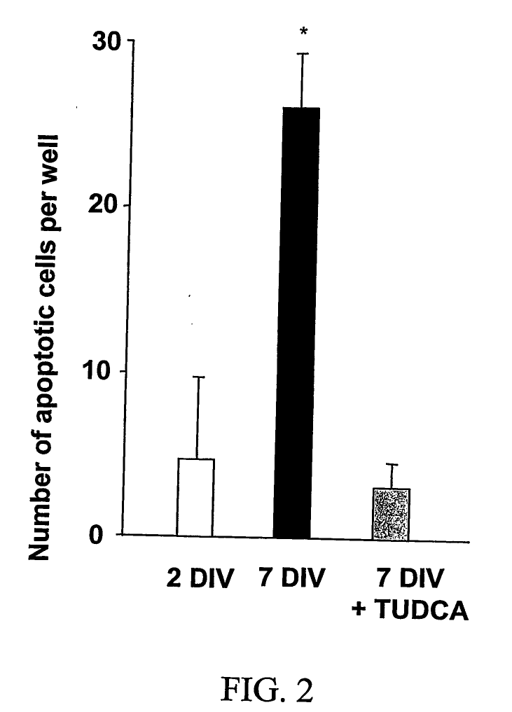

Apoptosis. As illustrated in FIG. 2, the number of TUNEL-positive cells was significantly higher in 7 DIV control cultures than that in 2 DIV control and 7 DIV TUDCA-treated cultures (p<0.01, one-factor ANOVA with post-hoc Scheffé's F-test).

Transplantation Experiment

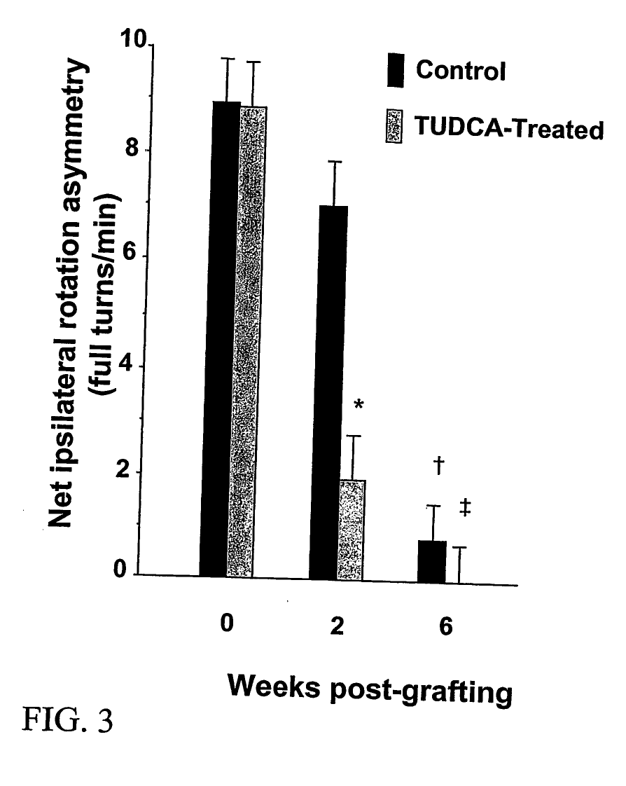

Rotational behavior. The amphetamine-induced rotation asymmetry scores are summarized in FIG. 3. At 2 weeks after transplantation, 4 of 6 TUDCA-treated rats exhibited at least 50% reduction in net motor asymmetry when compared to pre-transplantation values. In contrast, none of the control animal showed 50% reduction in motor asymmetry. At 6 weeks following transplantation, all rats of both the control and the TUDCA-treated groups exhibited>50% reduction in motor asymmetry scores.

A two-factor ANOVA revealed a significant Group×Time interaction of the net rotational scores over the testing period (F_{(1, 2)}=43.83, p<0.001), indicating that the TUDCA-treated rats exhibited a more rapid behavioral recovery than the control rats. Indeed, the mean net rotation values were significantly reduced 2 weeks after grafting when compared to pregrafting values in the TUDCA-treated group (p<0.01, paired Student's t test) but not in the control group (p>0.05). In addition, a one-factor ANOVA with post-hoc Scheffé's F-test showed that the TUDCA-treated group exhibited lower net asymmetry values than the control group 2 weeks after transplantation (F_{(1, 10)}=5.51, p<0.05). At 6 weeks after surgery, both groups had significantly reduced motor asymmetry scores (paired Student's t-test,p<0.05) and there was not longer any difference between the two groups (one-factor ANOVA, p>0.05).

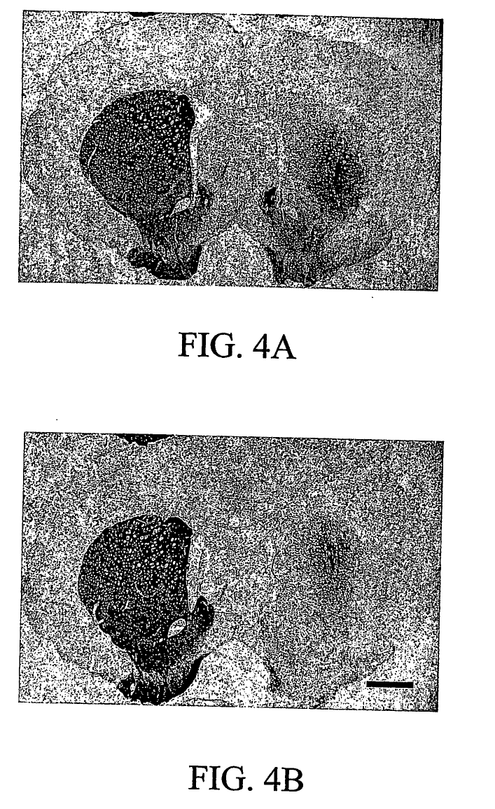

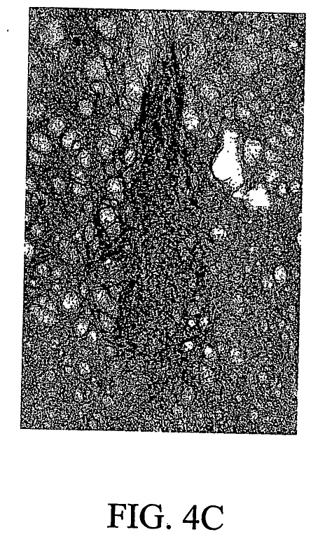

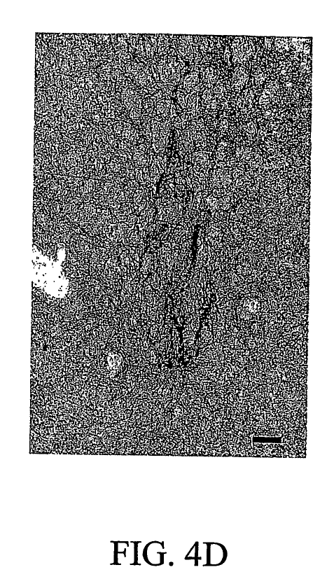

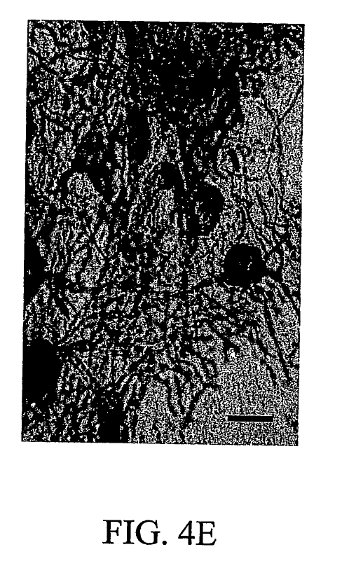

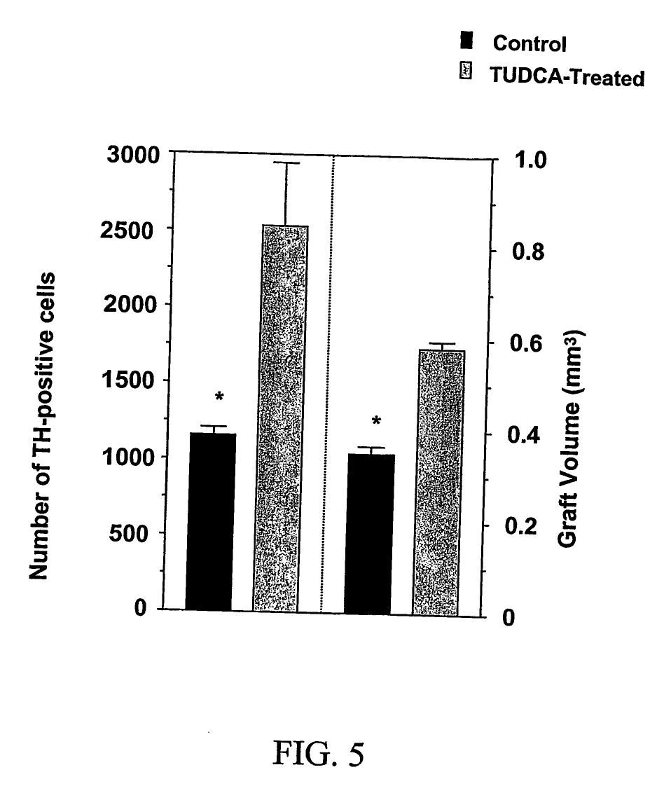

Graft survival. The majority of transplants was discernible in the center of striatum and oval-shaped. They contained numerous TH-immunoreactive neurons and fibers (FIG. 4). A few of transplants were misplaced into the overlying corpus callosum and the frontal cortex along the cannula tracks. FIG. 5 summarizes the mean number of TH-immunoreactive neurons in the grafts in the control and the TUDCA-treated groups 6 weeks after grafting. The mean number of TH-immunoreactive neurons in the grafts was significantly greater in the TUDCA-treated group than in the control group. More than a three-fold increase in the mean number of TH-immunoreactive neurons in the grafts was observed in the TUDCA-treated group when compared to the control group (one-factor ANOVA test followed by a post-hoc Scheffé's F-test, F_{(1, 10)}=6.65, p<0.05). The graft volume was also significantly larger in the TUDCA-treated group than that in the control group (p<0.05, one-factor ANOVA test followed by a post-hoc Scheffé's F-test, F_{(1, 10)}=8.22). At 6 weeks after transplantation, the grafts in the control animals exhibited a typical morphology of nigral grafts. The majority of TH-immunopositive neurons were located at the periphery of the grafts, leaving the center of the grafts relatively devoid of TH-immunoreactivity (FIG. 4D). However, it seemed that the grafts in the TUDCA-treated animals lacked this typical morphology of nigral grafts at this time-point. Most TUDCA-treated animals showed an even distribution of TH-immunoreactive neurons in the graft area (FIG. 4C). The TH-immunoreactive neurons possessed multipolar cell bodies with several clearly stained neurites (FIG. 4E). The areas of the host striatum that were reinnervated by the grafts were found to be larger in the TUDCA-treated group than in the control group (FIGS. 4A and B).

Apoptosis

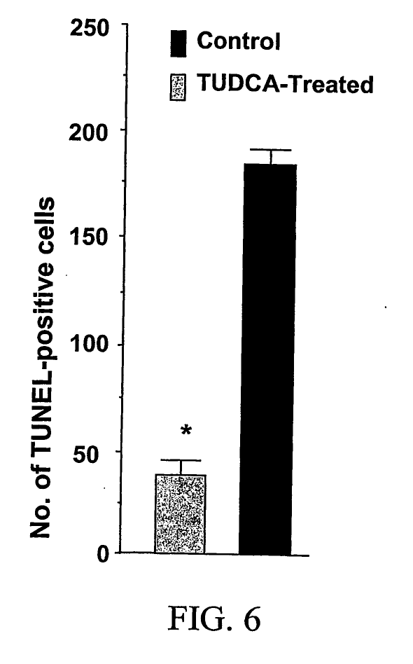

The mean number of TUNEL-positive cells in the grafted areas is summarized in FIG. 6 for the control and TUDCA-treated groups 4 days post-transplantation. The number of apoptotic cells was significantly smaller in the TUDCA-treated group than in the control group (p<0.01, one-factor ANOVA test followed by a post-hoc Scheffé's F-test, F_{(1, 10)}=20.06). A large number of apoptotic cells with DNA fragmentation in nuclei were clustered and located within the grafts in the control group (data not shown). Only a few apoptotic cells were observed in several patches within the grafts in the TUDCA-treated group (data not shown). Occasionally, a few scattered apoptotic cells were found in surrounding host tissue of the grafts in both groups.

The above examples show that the application of TUDCA facilitates to the survival of dopamine neurons in vitro and in vivo. In addition, TUDCA can significantly reduce apoptosis in VM tissue cultures and within the transplants, suggesting that TUDCA exerts beneficial effects on dopamine neuron survival mainly through anti-apoptotic mechanisms. The three-fold improvement of graft survival in the TUDCA-treated group leads to a rapid recovery of behavioral asymmetry when compared to the control group. These results confirm and provide further supportive evidence to the non-binding theory that apoptosis is a contributor to the loss of dopamine neurons. This dramatic cell loss can be prevented if an anti-apoptotic agent is applied to the tissue preparation or immediately after grafting.

The culture system where neuronal death was induced by serum deprivation in mesencephalic cultures is well known as an in vitro model to induce apoptosis. This in vitro model has been used extensively to examine the effects of anti-apoptotic agents and neurotrophic factors on apoptosis that occurs in the VM tissue cultures and the survival of dopamine neurons (R. L. Branton et al., Exp. Neurol. 160:88-98; 1999; E. D. Clarkson et al., Neuroreport 7:145-149; 1995; E. D. Clarkson et al., Cell Tissue Res. 289:207-210; 1997; G. S. Schierle et al., Nat. Med. 5:97-100; 1999; W. M. Zawada et al., Exp. Neurol. 140:60-67; 1996). The data presented above shows that there was cell loss of dopamine neurons after the culture medium was switched to serum-free conditions. The TUNEL assay results suggest that the majority of the cell loss may possibly result from apoptosis. The data also indicate that the addition of TUDCA to the cultures leads to a reduction in the number of apoptotic cells and an increase in the number of dopamine neurons, indicating TUDCA exerts neuroprotective effects on dopamine neurons mainly through anti-apoptotic mechanisms.

Earlier studies have shown that around 80-95% of grafted DA neurons die following transplantation (P. Brundin et al., Prog. Brain Res. 71:293-308, 1987; N. Nakao et al., Proc. Natl. Acad. Sci. USA 91:12408-12412, 1994; H. Sauer et al., Restor. Neurol. Neurosci. 2:123-135, 1991; H. Sauer et al., Exp. Brain Res. 90:54-62, 1992; G. S. Schierle et al., Nat. Med. 5:97-100, 1999; A. Zuddas et al., Eur. J. Neurosci. 3:72-85, 1991). Cell death in neural grafts has recently been found to occur in the four phases during the transplantation procedure (P. Brundin et al., Cell Transplant. 9:179-195; 2000.). Nevertheless, evidence shows that the majority of cell loss occurs within the first few days after transplantation (W.-M. Duan et al., Exp. Brain Res. 104:227-242, 1995; M. Emgard et al., Exp. Neurol. 160, 279-288, 1999; G. S. Schierle et al., Nat. Med. 5:97-100, 1999; W. M. Zawada et al., Brain Res. 786, 96-103; 1998).

Several factors have been demonstrated to contribute to this cell death. Mechanical damage, anoxia and nutritional insufficiency during the transplantation procedure may lead to immediate cell death and production of reactive oxygen species. A variety of free radicals can subsequently cause transplanted dopamine neurons to undergo apoptotic cell death. Nigral grafts that are transplanted into the striatum are heterotopic grafts. The new environment in the striatum may not favor nigral graft survival and the neural grafts may lack sufficient neurotrophic support. TUDCA supplemented medium used during the preparation of cell suspension increased the survival of neural grafts by approximately three-fold and the graft function was also improved. Although not wishing to be bound by theory, these data provide evidence that the majority of cell loss in the grafts is closely related to apoptosis (G. S. Schierle et al., Nat. Med. 5:97-100, 1999; W. M. Zawada et al., Brain Res. 786, 96-103; 1998). In addition, antioxidants and anti-apoptotic agents, such as TUDCA, could be used in combination to provide additional effects in preventing cell death of transplanted nigral grafts.

The early onset of behavioral effects occurs in the treated group and all the rats exhibited>50% reduction of net motor asymmetry. Although the mechanisms behind rapid onset of behavioral recovery need to be further evaluated, one possible, but non-limiting, explanation could be that the administration of these anti-apoptotic agents and neurotrophic factors facilitates the rapid maturation of transplanted dopamine neurons. In addition, previous studies have demonstrated that a certain number of intrastriatal TH-positive grant neurons is required to induce a behavioral recovery of 50% and a plateau recovery level of slightly over 100% is found in the range above 2000 intrastriatal TH-positive graft neurons. This phenomena may reflect the fact that when a certain number of dopamine neurons survives after transplantation at an early time-point, a similar number of transplanted dopamine neurons will remain for later time-points (W.-M. Duan et al., Exp. Brain Res. 104:227-242, 1995; W.-M. Duan et al., Neuroscience 100:521-530, 2000). These observations highlight the value of applying neuroprotective strategies to prevent cell loss in neural grafts at early time-points after transplantation rather than at later time-points. Another possible explanation for the apparent discrepancy between cell number and behavior is that a level of dopamine innervation is required to induce a behavioral correction. This level is achieved at an earlier time point in the grafts treated with TUDCA since there is a greater survival of the grafted cells. As the non-TUDCA treated cells begin to elaborate additional processes and innervate the host striatum, they too will eventually provide a level of innervation that is capable of correcting the locomotor asymmetry, but on a delayed time scale compared to the cells treated with TUDCA.

Caspase activity is believed to play a role in apoptosis (G. S. Salvesen et al., Cell 91:443-446, 1997). A pathway for caspase activation involves the mitochondria (D. R. Green et al., Science 281:1309-1312, 1998; G. Kroemer et al., Nat. Med. 6:513-519, 2000). TUDCA has been shown to prevent mitochondrial swelling and disruption of the outer mitochondrial membrane. Membrane stability can inhibit pro-apoptotic molecules, cytochrome c release and lead to changes in cytochrome c-mediated downstream events, such as caspase activity. It is believed that TUDCA significantly reduces 3-nitropropionic acid (3-NP)-mediated neuronal cell death in striatal tissue cultures. In addition, the systemic administration of TUDCA can reduce striatal degeneration mainly through anti-apoptotic mechanisms and ameliorate neurological deficits in a 3-NP-lesioned rat model of Huntington's disease (C. D. Keene et al., Exp. Neurol. 171; 351-360, 2001).

As the majority of transplanted dopamine neurons die immediately following transplantation, an effort has been focused on the enhancement of graft survival to obtain optimum graft benefit. Indeed, numerous experimental studies have shown that the extent of behavioral recovery strongly correlates with the graft survival (P. Brundin et al., In: S. B. Dunnett and A. Bjorklund, eds., Functional neural transplantation, New York: Raven Press; 1994:9-46; N. Nakao et al., Proc. Natl. Acad. Sci. USA 91:12408-12412,1994; H. Sauer et al., Exp. Brain Res. 90:54-62, 1992). Furthermore, clinical studies with neural transplantation in Parkinson's disease have also demonstrated that the extent of symptomatic improvement after transplantation is closely related to the degree of neural graft survival and graft reinnervation (P. Brundin et al., Brain 123:1380-1390, 2000; J. H. Kordower et al., J. Comp. Neurol. 370:203-230, 1996; P. Piccini et al., Nat. Neurosci. 2:1137-1140, 1999). A large number of transplanted dopamine neurons generally results in a more pronounced clinical improvement. However, a recent work by Freed et al. spurred controversy regarding the requirement of the number of transplanted dopamine neurons for clinical improvement (P. Brundin et al., Nat. Med. 7:512-513, 2001; S. B. Dunnett et al., Nat. Rev. Neurosci. 2:365-369, 2001; C. R. Freed et al., N. Engl. J. Med. 344:710-719, 2001). In their double-blind placebo-controlled clinical trial of embryonic tissue transplants for Parkinson's disease, they reported that a small proportion of transplanted subjects eventually developed dystonia and dyskinesias and claimed that these side effects might be due to the continued fiber outgrowth in the transplantation sites, leading to the production of a relative excess of dopamine in subjects' brain. They therefore proposed to transplant less tissue to solve this problem in the future clinical trials. However, only modest numbers of dopamine neurons were reported to survive in the two post mortem autopsy cases in the study. In addition, other factors, for example, the tissue preparation, transplantation technique and graft location may contribute to the occurrence of transplantation related side effects.

As another example, TUDCA was also effective in reducing apoptosis of pancreatic islet cells after 2 days in vitro. The number of TUNEL-positive cells was approximately 29 in non-treated controls, but only 12 in cells exposed to TUDCA. The more than 50% reduction by apoptosis by TUDCA was achieved after exposure of cells to TUDCA during the isolation procedure, followed by incubation with the bile acid during culture in vitro.

The complete disclosure of all patents, patent documents, and publications cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of promoting viability of a transplant cell population comprising contacting the transplant cell population with an effective amount of a compound selected from the group consisting of a hydrophilic bile acid, a salt thereof, an analog thereof, and a combination thereof.

2. The method of claim 1 wherein cells of the transplant cell population are differentiated cells.

3. The method of claim 1 wherein cells of the transplant cell population are precursor cells.

4. The method of claim 1 wherein the contacting occurs in vitro.

5. The method of claim 1 wherein the contacting occurs in vivo.

6. The method of claim 5 wherein the contacting occurs in a donor of the transplant cell population.

7. The method of claim 1 wherein contacting the transplant cell population with the compound comprises contacting the transplant cell population with the compound prior to transplanting the transplant cell population in a subject.

8. The method of claim 7 wherein the subject is a human.

9. The method of claim 7 further comprising treating the subject with the compound.

10. The method of claim 9 wherein treating the subject with the compound comprises administering the compound to the subject prior to transplanting the transplant cell population in the subject.

11. The method of claim 10 wherein treating the subject with the compound comprises administering the compound to the subject after transplanting the transplant cell population in the subject.

12. The method of claim 9 wherein treating the subject with the compound comprises administering the compound to the subject after transplanting the transplant cell population in the subject.

13. The method of claim 9 wherein treating the subject comprises treating the subject parenterally with the compound.

14. The method of claim 9 wherein treating the subject comprises treating the subject orally with the compound.

15. The method of claim 1 wherein contacting the transplant cell population with the compound comprises contacting the transplant cell population in combination with a pharmaceutically acceptable carrier.

16. The method of claim 1 wherein the cells of the transplant cell population comprise autologous cells, heterologous cells, or xenologous cells.

17. The method of claim 1 wherein the cells of the transplant cell population comprises at least a portion of autologous tissue, heterologous tissue, or xenologous tissue.

18. The method of claim 1 wherein the transplant cell population is an organ.

19. The method of claim 18 wherein the organ is a liver, a kidney, a heart, a lung, or a pancreas.

20. The method of claim 18 further comprising treating the subject with the compound after transplanting the transplant cell population in the subject.

21. The method of claim 1 further comprising treating a subject with the compound after transplanting the transplant cell population in the subject.

22. A method for treating a human having Parkinson's disease, the method comprising contacting a transplant cell population in vitro with an effective amount of tauroursodeoxycholic acid in combination with a pharmaceutically acceptable carrier, wherein the transplant cell population are differentiated cells; and
transplanting the transplant cell population into the human.

23. A method for treating a subject having Parkinson's disease, the method comprising contacting a transplant cell population with an effective amount of a compound selected from the group consisting of ursodeoxycholic acid, a salt thereof, an analog thereof, and a combination thereof to promote viability of the transplant cell population; and
transplanting the transplant cell population into a subject.

24. The method of claim 23 wherein the ursodeoxycholic acid analog includes a conjugated derivative.

25. The method of claim 24 wherein the conjugated derivative is tauroursodeoxycholic acid.

26. The method of claim 23 wherein the subject is a human.

27. The method of claim 23 wherein the cells are differentiated cells.

28. The method of claim 23 wherein the cells are precursor cells.

29. The method of claim 23 wherein the contacting occurs in vitro.

30. The method of claim 23 wherein the contacting occurs in vivo.

31. The method of claim 30 wherein the contacting occurs in a donor of the transplant cell population.

32. The method of claim 23 wherein contacting the transplant cell population with the compound comprises contacting the transplant cell population with the compound prior to transplanting the transplant cell population in the subject.

33. The method of claim 32 further comprising treating the subject with the compound.

34. The method of claim 33 wherein treating the subject with the compound comprises administering the compound to the subject prior to transplanting the cells in the subject.

35. The method of claim 34 wherein treating the subject with the compound comprises administering the comprising to the subject after transplanting the cells in the subject.

36. The method of claim 33 wherein treating the subject with the compound comprises administering the compound to the subject after transplanting the cells in the subject.

37. The method of claim 23 wherein treating the subject comprises treating the subject parenterally with the compound.

38. The method of claim 23 wherein treating the subject comprises treating the subject orally with the compound.

39. The method of claim 23 wherein contacting the transplant cell population with the compound comprises contacting the transplant cell population in combination with a pharmaceutically acceptable carrier.

40. The method of claim 23 wherein the cells comprise autologous cells, heterologous cells, or xenologous cells.

41. The method of claim 23 wherein the cells comprises at least a portion of autologous tissue, heterologous tissue, or xenologous tissue.

42. A method, comprising:
treating a donor of a transplant cell population with a compound selected from the group consisting of a hydrophilic bile acid, a salt thereof, an analog thereof, and a combination thereof.

43. The method of claim 42 wherein the hydrophilic bile acid comprises ursodeoxycholic acid, and treating the donor comprises treating the donor with the ursodeoxycholic acid.

44. The method of claim 42 wherein the hydrophilic bile acid comprises tauroursodeoxycholic acid, and treating the donor comprises treating the donor with the tauroursodeoxycholic acid.

45. The method of claim 42 wherein the donor of the transplant cell population is living.

46. The method of claim 42 wherein treating the donor comprises treating the donor with the compound before removal of the transplant cell population from the donor.

47. The method of claim 42 wherein treating the donor comprises treating the donor with the compound during removal of the transplant cell population from the donor.

48. The method of claim 42 wherein treating the donor comprises treating the donor with the compound after removal of the transplant cell population from the donor.

49. The method of claim 42 wherein the transplant cell population is at least a portion of an organ of the donor.

50. The method of claim 49 wherein the at least a portion of the organ is the entire organ of the donor.

51. The method of claim 42 wherein treating the donor comprises treating the donor parenterally with the compound.

52. The method of claim 42 wherein treating the donor comprises treating the donor orally with the compound.

53. The method of claim 42 wherein treating the donor with the compound comprises contacting the transplant cell population in combination with a pharmaceutically acceptable carrier.

54. A method, comprising:
treating a subject for a transplant cell population with a compound selected from the group consisting of a hydrophilic bile acid, a salt thereof, an analog thereof, and a combination thereof.

55. The method of claim 54 wherein the hydrophilic bile acid comprises ursodeoxycholic acid, and treating the subject comprises treating the subject with the ursodeoxycholic acid.

56. The method of claim 54 wherein the hydrophilic bile acid comprises tauroursodeoxycholic acid, and treating the subject comprises treating the subject with the tauroursodeoxycholic acid.

57. The method of claim 54 wherein treating the subject comprises treating the subject with the compound before transplanting the transplant cell population into the subject.

58. The method of claim 54 wherein treating the subject comprises treating the subject with the compound during transplanting the transplant cell population into the subject.

59. The method of claim 58 wherein treating the subject comprises treating the transplant cell population in the subject.

60. The method of claim 54 wherein treating the subject comprises treating the subject with the compound after transplanting the transplant cell population into the subject.

61. The method of claim 54 wherein the transplant cell population is at least a portion of an organ.

62. The method of claim 61 wherein the at least a portion of the organ is the entire organ.

63. The method of claim 54 wherein treating the subject comprises treating the subject parenterally with the compound.

64. The method of claim 54 wherein treating the subject comprises treating the subject orally with the compound.

65. The method of claim 54 wherein treating the subject with the compound comprises contacting the transplant cell population in combination with a pharmaceutically acceptable carrier.
